(19) United States
(12) Patent Application Publication
(10) Pub. No.: US 2015/0086186 A1
(43) Pub. Date: Mar. 26, 2015

(54) HANDHELD ELECTRONIC VAPORIZATION DEVICE

(71) Applicant: 9208-8699 Quebec Inc., Saint-Paul-de-Montmagny (CA)

(72) Inventor: Grégoire Boki, Saint-Paul-de-Montmagny (CA)

(21) Appl. No.: 14/386,622

(22) PCT Filed: Feb. 25, 2013
(86) PCT No.: PCT/CA2013/000170

§ 371 Date: Sep. 19, 2014

Related U.S. Application Data
(60) Provisional application No. 61/613,724, filed on Mar. 21, 2012.

(51) Int. Cl. A61M 11/04; A61M 15/00

(52) U.S. Cl. CPC A61M 11/042; A61M 15/0001
USPC 392/387; 392/386

(57) ABSTRACT

A method and related handheld electronic vaporization device comprising a heating chamber adapted to receive a removable capsule, at least one air preheating chamber and at least one fluid communication passageway from exterior of the device, through the at least one preheating chamber, the heating chamber and the capsule. The heating chamber is adapted to receive the capsule having an outlet and being adapted to hold a substrate containing a volatile compound that, when heated, yields vapor including the volatile compound. The at least one preheating chamber is in contact with the heating chamber. At least one of the heating chamber or the at least one preheating chamber is electrically heated. When negative air pressure is applied to the capsule's outlet, air from the exterior of the device is allowed in the at least one fluid communication passageway to the capsule's outlet.

### TECHNICAL FIELD

The present invention relates to heating and vaporization devices. More specifically, the present invention relates to a device for converting to a gaseous phase organo-volatile compounds contained in a substrate prior to their inhalation, e.g., for medicinal purpose.

### BACKGROUND

Many means are presently known for converting to a gaseous phase organo-volatile compounds contained in a substrate. These means can be divided into two categories.

A first type of device is based on the injection of a stream of hot air through the substrate to heat and vaporize the organo-volatile compounds.

One exemplary drawback of this type of device is that it tends to dilute the vapors of organo-volatile compounds into the stream of heating air.

A second type of device is based on the heating of the walls of a receptacle supporting the substrate, which is heated through the walls faces.

One exemplary drawback of this type of device is that it tends to heat the substrate exterior more than the interior.

### SUMMARY

A first aspect of the present invention is directed to a handheld electronic vaporization device comprising a heating chamber adapted to receive a removable capsule, at least one air preheating chamber and at least one fluid communication passageway from exterior of the device, through at least the air preheating chamber, to the removable capsule adapted to the heating chamber. The heating chamber is adapted to receive the removable capsule having an outlet and being adapted to hold a substrate containing a volatile compound that, when heated, yields vapor including the volatile compound. At least one of the heating chamber or air preheating chamber is electrically heated. When negative air pressure is applied to the removable capsule's outlet, air from the exterior of the device is allowed in the at least one fluid communication passageway to the removable capsule's outlet.

Optionally, the at least one air preheating chamber may comprise a plurality of concentric air preheating chambers, at least one of which being in contact with the heating chamber. The heating chamber may optionally be made of at least one material selected in the group consisting of plastic, ceramics and metals. Optionally, the heating chamber and the at least one air preheating chamber may be electrically heated using a preset electric heat power setting or using an adjustable electric heat power setting. The at least one air preheating chamber may also be heated so as to allow heat therefrom to be transferred to the air from the exterior of the device. The heating chamber and the at least one air preheating chamber may be of different configurations. The handheld electronic vaporization device may further comprise at least one electric element to directly heat the heating chamber while heating indirectly the at least one air preheating chamber positioned adjacent to the heating chamber, e.g., through radiation and air contact. Optionally, the at least one air preheating chamber may comprise a plurality of chambers of different length, distance and configuration in relation to heating chamber. The at least one air preheating chamber may also comprise a plurality of chambers that are eccentric in relation to the heating chamber. The at least one air preheating chamber may also comprise a plurality of chambers that are located unilaterally in relation to the heating chamber. The plurality of chambers may comprise more than 2 chambers. The at least one air preheating chamber may also comprise a tubular section coiled around the heating chamber.

A second aspect the present invention is directed to a method for drawing vapor from a handheld electronic vaporization device. The handheld electronic vaporization device comprises a heating chamber adapted to receive a removable capsule having a vapor outlet. The removable capsule is adapted to hold a substrate of volatile compound. The handheld electronic vaporization device further comprises at least one air preheating chamber in contact with the heating chamber and at least one electric heating element in contact with the heating chamber or at least one air preheating chamber. A fluid passageway is present from exterior of the device to the at least one preheating chamber to the heating chamber to the removable capsule. The method comprises applying electrical power to the heating element for heating the removable capsule in order to yield vapor including the volatile compound and heating the at least one air preheating chamber. The method also comprises applying a negative air pressure to the vapor outlet of the removable capsule causing air from the exterior of the device to enter the fluid passageway and successively pass through the at least one air preheating chamber, the heating chamber and the removable capsule to reach the vapor outlet. The air from the exterior is preheated in the at least one preheating chamber prior to entering the removable capsule.

### BRIEF DESCRIPTION OF THE DRAWINGS

In the appended drawings:

FIG. 1 is a top plan view of an exemplary electronic handheld vaporization device according to a first illustrative embodiment of the present invention;

FIG. 2 is a cross-section taken along line A-A from FIG. 1, the exemplary handheld vaporization device with removable substrate capsule retracted from heating body.

FIG. 3 is a large scale cross-section taken along line 2-2 from FIG. 1 of the exemplary electronic handheld vaporization device heating body in view of detailing heating chamber, air preheating chambers and fluid communication pathways.

FIG. 4 is a flow chart of an exemplary use of the exemplary handheld vaporization device depicted in FIGS. 1, 2 and 3.

### DETAILED DESCRIPTION

A first aspect of the present invention is directed to provide improved method and system for converting to a gaseous phase organo-volatile compounds contained in a substrate. A second aspect of the invention is directed a portable system for converting to a gaseous phase organo-volatile compounds contained in a substrate.

More specifically, in accordance with the present invention, there is provided an electronic handheld vaporization device comprising at least one of a heating chamber or air preheating chamber that are heated by electric power in order to heat a substrate of volatile compound contained in a removable capsule so as to yield vapor including the volatile compound. The heating chamber, the at least one air preheating chamber and the capsule comprise at least one inlet and outlet so as to create a successive fluid communication path from exterior of device to the capsule outlet for releasing the vapor that includes the volatile compound.

The removable capsule is made to hold a substrate of organo-volatile compound and may be made to transfer heat to the substrate via pre heated air from the one or more air preheating chamber, which is in contact with the heating chamber (e.g., through its walls).

By applying heat to the handheld vaporization device's heating chamber (e.g., through one or more electrically powered element), heat is transferred to the substrate and also to the air being routed through a passageway running from the exterior of the device towards the capsule. This may provide exemplary advantages of creating a uniform heat across the substrate present into the capsule and permitting extraction of undiluted organo-volatile elements.

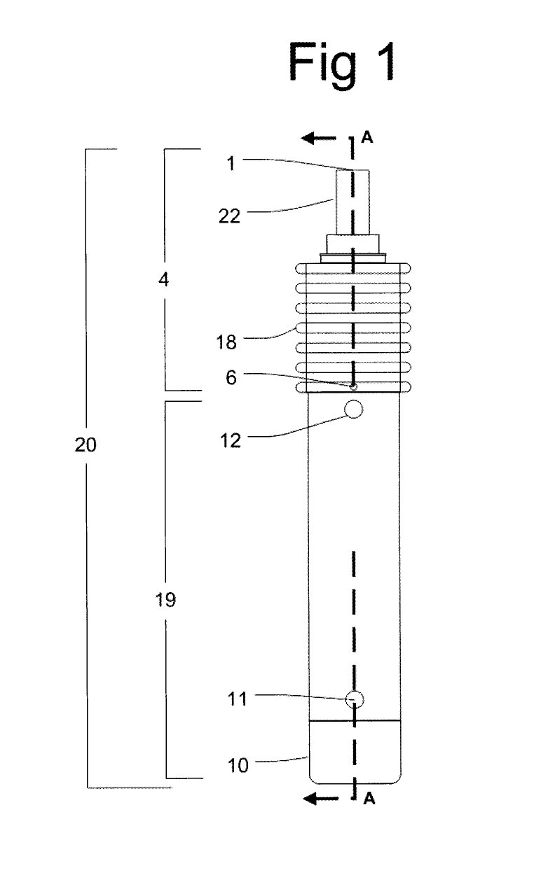

An electronic handheld vaporization device 20 according to a first illustrative embodiment of the present invention will now be described with reference to FIGS. 1, 2 and 3.

The electronic handheld vaporization device 20 comprises a first elongated body 19 defining the handle of the device 20. The body 19 comprises a charging indicator light 11, a temperature indicator light 12 and a temperature adjustment knob 10. The electronic handheld vaporization device 20 also comprises a second elongated body defining a heating head 4 including temperature ribs 18, an air inlet 6 and a vaporization capsule 22.

The first elongated body 19 of the handheld device 20 comprises an upper section and a lower section. The upper section comprises a cavity therein defining a container for an electronic control 7, an electric power accumulator 8 and a rear electronic board 9. The lower section comprises a temperature adjustment knob 10. The temperature adjustment knob 10 is shown on the example of FIGS. 1, 2 and 3 to be angularly mobile in relation to the upper section on the longitudinal axis to the device 20 such as to permit angular movement to control position of an electronic graduator located on the electronic board 9.

The electronic control 7 may comprise an indicator light 12 (e.g., to inform user of operation temperature). The indicator light 12 may be used to inform user by coded light sequence. The electronic control 7 may additionally include sound speaker to inform the user by coded sound sequence.

The electric power accumulator 8 may be of various types including but not limited to li-on type battery. The electric power accumulator 8 may also be a direct power source originating from a domestic power line (e.g., when the device 20 apparatus is plugged into wall outlet (not shown)).

The rear electronic board 9 may include an indicator light 11, a battery charger (not shown) and an electronic potentiometer (not shown). The indicator light 11 may transmit information to the user via various coded signals.

The temperature adjustment knob 10 may have various shape and be made of various materials. The angular movement of the potentiometer may vary (not shown).

The second elongated body defining a heating head 4 of the handheld device 20 comprises a cavity containing a first heating body 14 and a second heating body 16. The heating head 4 additionally comprises an air inlet 6, temperature ribs 18 and a capsule seat opening 21.

The heating head 4 may be of various size and shape inasmuch to stay portable. The heating head 4 may be made of various non conductive and heat resistant materials such as, but not limited to plastics, wood and ceramic. The temperature ribs 18 may be of various size and configuration in way to keep temperature safe in case of skin contact by the user.

The air inlet 6 may be of various size, configuration or position inasmuch to permit air to enter into a first air preheating chamber 13.

The capsule seat opening 21 may be of various dimension and configuration in way to adequately receive and a capsule tip 23.

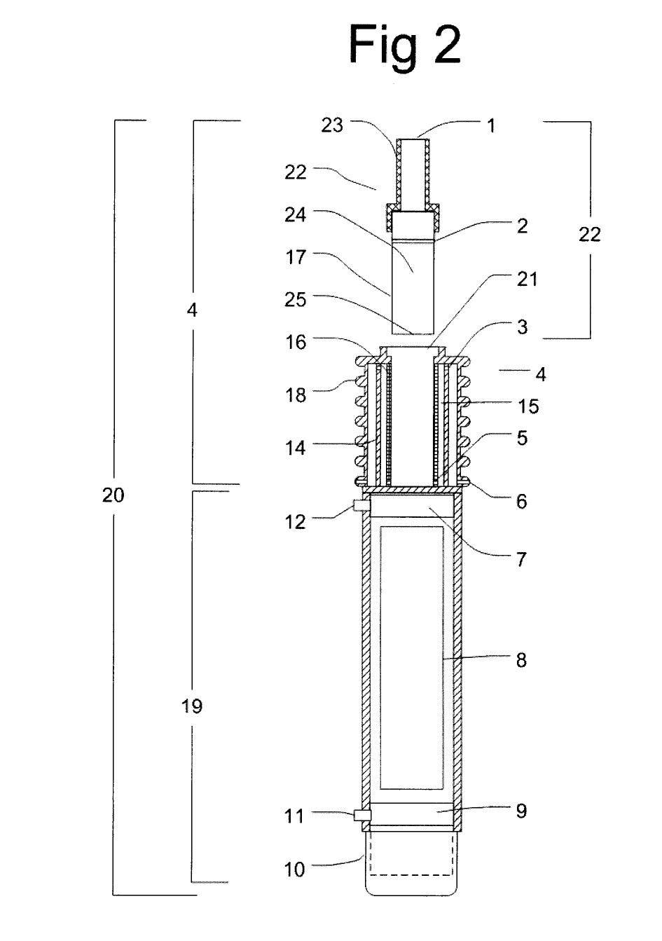

The capsule 22, shown on FIG. 2 removed from the second heating body 16, comprises a capsule body 24, a substrate mesh 2 and the capsule tip 23.

The capsule body 24 is defined by a capsule body wall 17. The capsule body wall 17 may be of various size, configuration and type of material so as to permit efficient heating of the substrate contained thereinto while in operation. A capsule body opening 25 is made to receive substrate load and permit heated air from a second airway 5 to enter thereinto.

The substrate mesh 2 may be of various size inasmuch as to keep particles from substrate in place while letting vapors created from heated substrate go through. The substrate mesh 2 may be of various area, material and position in relation to the capsule body opening 25.

The capsule tip 23 is a hollow body affixed to the capsule body 17 and defines a vapor outlet 1. The capsule tip 23 may be made of various heat resistant material and be of various shape and dimension so as to fit into the capsule seat opening 21. Length of the capsule tip 23 may be extended using tubing (not shown). The vapor outlet 1 is a fluid communication and may be of various size and dimensions.

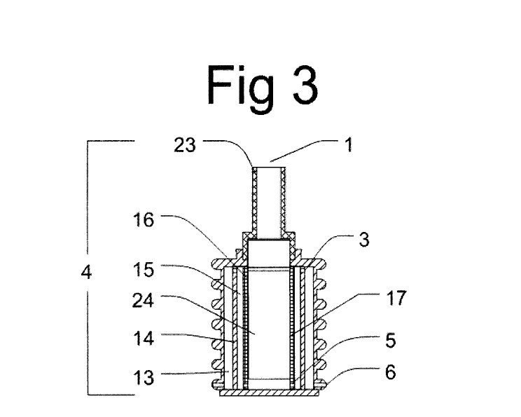

With reference to FIG. 3, the capsule 22 is in place within the heating head 4 and a fluid communication pathway is visible. At least one heating element is present (not shown) in the second heating body 16. Upon activation of the at least one heating element, heat is transmitted inwardly to the substrate body wall 17, which in turn transmits heat to the substrate contained in the cavity 21 defining the capsule body 24. Upon the activation of the at least one heating element, heat is also transmitted outwardly through a second air heating chamber 15, the first heating body 14, the first air heating chamber 13 and the heating body 4.

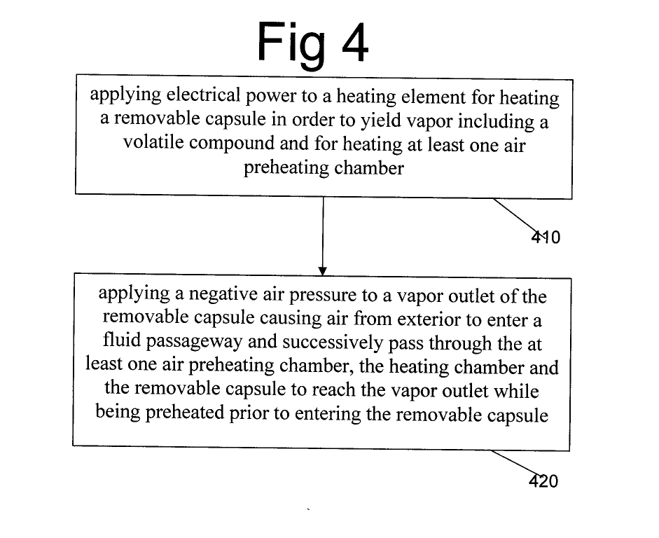

FIG. 4 shows a flow chart of an exemplary use of the exemplary handheld vaporization device 20 depicted in FIGS. 1, 2 and 3. In step 410, electrical power is applied to a heating element for heating substrate contained in a removable capsule in order to yield vapor including a volatile compound and for heating at least one air preheating chamber. In step 420, negative air pressure is applied to a vapor outlet of the removable capsule causing air from exterior to enter a fluid passageway and successively pass through the at least one air preheating chamber and the removable capsule contained in the heating chamber to reach the vapor outlet while being preheated prior to entering the removable capsule.

For instance, once a set point temperature is reached, a ready signal may be given to a user. The user may then apply a negative air pressure to the vapor outlet 1, thereby drawing vapor therefrom. The vacuum created at the vapor outlet 1 is transmitted via the fluid pathway constituted by the capsule opening 25, the second airway 5, the second air heating chamber 15, the first airway 3, the first air heating chamber 15 and the air inlet 6. Depressurization at air inlet 6 let air into the first air heating chamber 13. The air heating chamber 13 being hot, air circulating therethrough is heated. In turn, heated air from the first air heating chamber 13 runs through the first airway 3 into the second heating chamber 15. The second heating chamber also being hot further heats air circulating therethrough. Thus, preheated air eventually gets to the second airway 5 to the substrate contained in the capsule body 24, via the capsule opening 25.

The substrate being heated by direct contact with the heated capsule body wall 17 receives extra heat from the preheated air entering thereinto. The example provides the exemplary advantage of enhanced extraction of organo-volatile compounds contained thereinto.

Other aspects, advantages and features of the present invention will become more apparent upon reading the following non restrictive description of illustrated embodiments thereof, given by way of example only with reference to the accompanying drawings. A method is generally conceived to be a self-consistent sequence of steps leading to a desired result. These steps require physical manipulations of physical quantities. Usually, though not necessarily, these quantities take the form of electrical or magnetic signals capable of being stored, transferred, combined, compared, and otherwise manipulated. It is convenient at times, principally for reasons of common usage, to refer to these signals as bits, values, parameters, items, elements, objects, symbols, characters, terms, numbers, or the like. It should be noted, however, that all of these terms and similar terms are to be associated with the appropriate physical quantities and are merely convenient labels applied to these quantities. The description of the present invention has been presented for purposes of illustration but is not intended to be exhaustive or limited to the disclosed embodiments. Many modifications and variations will be apparent to those of ordinary skill in the art. The embodiments were chosen to explain the principles of the invention and its practical applications and to enable others of ordinary skill in the art to understand the invention in order to implement various embodiments with various modifications as might be suited to other contemplated uses.

1. A handheld electronic vaporization device comprising:
a heating chamber adapted to receive a removable capsule, the removable capsule having an outlet and being adapted to hold a substrate of volatile compound that, when heated, yields vapor including the volatile compound;
at least one air preheating chamber in contact with the heating chamber, wherein at least one of the heating chamber and the at least one air preheating chamber is electrically heated; and
at least one fluid communication passageway from exterior of the device, through at least one of the heating chamber and the at least one air preheating chamber, to the removable capsule whereby when negative air pressure is applied to the outlet of the removable capsule, air from the exterior of the device is allowed in the at least one fluid communication passageway to the outlet of the removable capsule.

2. A handheld electronic vaporization device as recited in claim 1, wherein the at least one air preheating chamber comprises a plurality of concentric air preheating chambers, at least one of which being in contact with the heating chamber.

3. A handheld electronic vaporization device as recited in claim 1, wherein the heating chamber is made of at least one material selected in the group consisting of plastic, ceramics and metals.

4. A handheld electronic vaporization device as recited in claim 1, wherein the heating chamber and at the least one air preheating chamber is electrically heated using a preset electric heat power setting.

5. A handheld electronic vaporization device as recited in claim 1, wherein the heating chamber and the least one air preheating chamber is electrically heated using an adjustable electric heat power setting.

6. A handheld electronic vaporization device as recited in claim 1, wherein the at least one air preheating chamber is heated so as to allow heat therefrom to be transferred to the air from the exterior of the device.

7. A handheld electronic vaporization device as recited in claim 1, wherein the heating chamber and the at least one air preheating chamber are of different configurations.

8. A handheld electronic vaporization device as recited in claim 1, further comprising at least one electric element directly heats the heating chamber.

9. A handheld electronic vaporization device as recited in claim 1, further comprising at least one electric element positioned adjacent to the at least one air preheating chamber indirectly heats the heating chamber.

10. A handheld electronic vaporization device as recited in claim 1, wherein the at least one air preheating chamber comprises a plurality of chambers of different length, distance and configuration in relation to heating chamber.

11. A handheld electronic vaporization device as recited in claim 1, wherein the at least one air preheating chamber comprises a plurality of chambers that are eccentric in relation to the heating chamber.

12. A handheld electronic vaporization device as recited in claim 1, wherein the at least one air preheating chamber comprises a plurality of chambers that are located unilaterally in relation to the heating chamber

13. A handheld electronic vaporization device as recited in claim 2, wherein the plurality of chambers comprise more than 2 chambers.

14. A handheld electronic vaporization device as recited in claim 1, wherein the at least one air preheating chamber comprises a tubular section coiled around the heating chamber.

15. A method for drawing vapor from a handheld electronic vaporization device comprising a heating chamber adapted to receive a removable capsule having a vapor outlet, the removable capsule being adapted to hold a substrate of volatile compound, wherein the handheld electronic vaporization device further comprises at least one air preheating chamber in contact with the heating chamber and at least one heating element in contact with the heating chamber and wherein a fluid passageway is present from exterior of the device to the at least one preheating chamber to the heating chamber to the removable capsule, the method comprising:
applying electrical power to the heating element for:
heating the removable capsule in order to yield vapor including the volatile compound; and
heating the at least one air preheating chamber;
applying a negative air pressure to the vapor outlet of the removable capsule causing air from the exterior of the device to enter the fluid passageway and successively pass through the at least one air preheating chamber, the heating chamber and the removable capsule to reach the vapor outlet, wherein the air from the exterior is preheated in the at least one preheating chamber prior to entering the removable capsule.
